(19) United States
(12) Patent Application Publication
(10) Pub. No.: US 2017/0368251 A1
(43) Pub. Date: Dec. 28, 2017

(54) Blood Isolation and Extraction Method and Device thereof

(71) Applicants: National University of Kaohsiung, Kaohsiung (TW); Biomimedtech Co., Ltd., Kaohsiung (TW); Neoasia Limited, Taipei City (TW)

(72) Inventors: Yi-Chang Chung, Kaohsiung (TW); Yi-Hong Chiu, Kaohsiung (TW); Chia-Chune Wu, Taipei City (TW); Che-Wen Chang, Taipei City (TW); Kai-Ming Wu, Kaohsiung (TW)

(21) Appl. No.: 15/193,083

(22) Filed: Jun. 26, 2016

(51) Int. Cl. A61M 1/34; B01D 39/16; B01D 11/04; A61M 1/36; B01D 39/08; C12N 5/078; B01D 39/14

(52) U.S. Cl. CPC A61M 1/3496; C12N 5/0644; C12N 5/0641; B01D 39/1692; B01D 39/14; A61M 1/3633; B01D 39/08; B01D 11/0492; B01D 39/083; A61M 2205/75

(57) ABSTRACT

A blood isolation and extraction method includes: providing a predetermined amount of blood; utilizing a platelet filter unit to filter the predetermined amount of blood to generate a filtered blood; utilizing a plasma separation unit to divide the filtered blood into a plasma layer and a blood cell layer for separating blood cells from blood plasma; and extracting the blood plasma from the plasma layer and the blood cells from the blood cell layer. In another embodiment, the blood isolation and extraction method further includes: providing a platelet-washing unit to wash the platelet filter unit with a solution to produce a platelet solution; and mixing the platelet solution with the blood plasma to produce a platelet and plasma mixed solution.

### BACKGROUND OF THE INVENTION

### 1. Field of the Invention

The present invention relates to a blood isolation and extraction method and device thereof. Particularly, the present invention relates to the blood isolation and extraction method and device thereof for separately extracting platelets, plasma and corpuscles.

### 2. Description of the Related Art

By way of example, Taiwanese Patent No. I395612, entitled “BLOOD SEPARATION METHOD,” discloses a conventional blood separation method. The conventional blood separation method includes the steps of: providing a filtering film having a flow channel and a plurality of holes and a receiving compartment connecting with the filtering film and communicating with the flow channel; actuating a flow of blood through the filtering film to substantially form a horizontal movement with respect to the filtering film; collecting the blood passing through the holes in the receiving compartment. The filtering film can generate a shear stress to block blood cells (i.e., hemocyte) so that the rest of blood (i.e., plasma) passes through the holes of the filtering film.

The filtering film has a first surface provided with a plurality of cambered surfaces while a second surface provided with a plurality of recessions. On the first surface each of the holes are formed among the cambered surfaces and are aligned with each of the recessions provided on the second surface. The holes have a diameter ranging 1-50 micrometer. The filtering film is made of a material of metal or metal alloy.

However, the filtering film applied in the blood separation method is only suitable for roughly separating blood cells with the 1-50 micrometer holes and cannot be successfully separate platelets from blood cells (i.e., red blood cells or white blood cells). Hence, there is a need of providing an improved blood separation method and system for extracting platelets from blood cells.

U.S. Pat. No. 6,893,412, entitled “PLATELET COLLECTING APPARATUS,” discloses a conventional platelet collecting apparatus. The platelet collecting apparatus comprises a centrifugal separator possessing a rotatable rotor, a first line for allowing the flow of the blood entering the centrifugal separator, a second line for allowing the flow of the blood emanating from the centrifugal separator, and a plasma collecting bag connected to the first line and the second line so as to collect the plasma emanating from the centrifugal separator and return the collected plasma to the centrifugal separator.

The platelet collecting apparatus further comprises a platelet collecting bag connected to the second line so as to collect the platelets emanating from the centrifugal separator, a blood delivering pump disposed in the first line, and a controller for controlling the operation of the rotor of the centrifugal separator and the operation of the blood delivering pump. The controller is provided with a function of varying the rotational frequency of the rotor during the course of blood collection in conformity with the amount of the blood entered into the centrifugal separator via the first line.

The platelet collecting apparatus must utilize the centrifugal separator to separate platelets from plasma in a centrifugal separation manner. However, the centrifugal separation manner results in lengthening a total processing time of blood separation and increasing a total cost and a total weight of blood separation apparatus. Hence, there is a need of providing an improved blood separation method and system for extracting platelets from blood cells. The above-mentioned patents are incorporated herein by reference for purposes including, but not limited to, indicating the background of the present invention and illustrating the situation of the art.

As is described in greater detail below, the present invention provides a blood isolation and extraction method and device thereof. A platelet filter unit is provided to filter a predetermined amount of blood to generate a filtered blood. The platelet filter unit is washed with a solution to produce a platelet solution which contains platelets remained on the platelet filter unit. A plasma separation unit is provided to divide the filtered blood into a plasma layer and a blood cell layer for separating blood cells from blood plasma in such a way as to improve the conventional blood separation method and device.

### SUMMARY OF THE INVENTION

The primary objective of this invention is to provide a blood isolation and extraction method. A platelet filter unit is provided to filter a predetermined amount of blood to generate a filtered blood. The platelet filter unit is washed with a solution to produce a platelet solution which contains platelets remained on the platelet filter unit. A plasma separation unit is provided to divide the filtered blood into a plasma layer and a blood cell layer for separating blood cells from blood plasma. Advantageously, the blood isolation and extraction method of the present invention is successful in rapidly separating the platelets and blood cells from the blood plasma.

The blood isolation and extraction method in accordance with an aspect of the present invention includes:

providing a predetermined amount of blood;

utilizing a platelet filter unit to filter the predetermined amount of blood to generate a filtered blood;

utilizing a plasma separation unit to divide the filtered blood into a plasma layer and a blood cell layer for separating blood cells from blood plasma; and

extracting the blood plasma from the plasma layer and the blood cells from the blood cell layer.

The blood isolation and extraction method in accordance with another aspect of the present invention includes:

providing a predetermined amount of blood;

utilizing a platelet filter unit to filter the predetermined amount of blood to generate a filtered blood;

washing or flushing the platelet filter unit with a solution to produce a platelet solution which contains platelets remained on the platelet filter unit;

utilizing a plasma separation unit to divide the filtered blood into a plasma layer and a blood cell layer for separating blood cells from blood plasma; and

mixing the platelet solution with the blood plasma retrieved from the plasma layer to produce a platelet and plasma mixed solution.

In a separate aspect of the present invention, the predetermined amount of blood is supplied by a blood supply unit or an injector.

In a further separate aspect of the present invention, the platelet filter unit includes a platelet adsorption net filter or a platelet adsorption plate.

In yet a further separate aspect of the present invention, the platelet adsorbing net filter includes a plurality of apertures and is made of platelet adsorption resin.

In yet a further separate aspect of the present invention, the platelet adsorbing plate is made of platelet adsorption resin.

In yet a further separate aspect of the present invention, the plasma separation unit includes a blood cell adsorbent, a blood cell adsorption material or a blood cell adsorption gel.

In yet a further separate aspect of the present invention, the plasma separation unit includes a tube to contain the plasma layer and the blood cell layer formed in the filtered blood.

Another objective of this invention is to provide a blood isolation and extraction device. A platelet filter unit is provided to filter a predetermined amount of blood to generate a filtered blood. The platelet filter unit is washed with a solution to produce a platelet solution which contains platelets remained on the platelet filter unit. A plasma separation unit is provided to divide the filtered blood into a plasma layer and a blood cell layer for separating blood cells from blood plasma. Advantageously, the blood isolation and extraction device of the present invention is successful in rapidly separating the platelets and blood cells from the blood plasma.

The blood isolation and extraction device in accordance with an aspect of the present invention includes:

a platelet filter unit provided to filter a predetermined amount of blood to generate a filtered blood; and

a plasma separation unit provided to divide the filtered blood into a plasma layer and a blood cell layer for separating blood cells from blood plasma;

wherein the blood plasma is retrieved from the plasma layer and the blood cells are retrieved from the blood cell layer.

The blood isolation and extraction device in accordance with another aspect of the present invention includes:

a platelet filter unit provided to filter a predetermined amount of blood to generate a filtered blood;

a platelet-washing unit provided to wash or to flush the platelet filter unit with a solution to produce a platelet solution which contains platelets remained on the platelet filter unit; and

a plasma separation unit provided to divide the filtered blood into a plasma layer and a blood cell layer for separating blood cells from blood plasma;

wherein the blood plasma is retrieved from the plasma layer and is further mixed with the platelet solution to produce a platelet and plasma mixed solution.

In a separate aspect of the present invention, the predetermined amount of blood is supplied by a blood supply unit or an injector.

In a further separate aspect of the present invention, the platelet filter unit includes a platelet adsorption net filter or a platelet adsorption plate.

In yet a further separate aspect of the present invention, the platelet adsorbing net filter includes a plurality of apertures and is made of platelet adsorption resin.

In yet a further separate aspect of the present invention, the platelet adsorbing plate is made of platelet adsorption resin.

In yet a further separate aspect of the present invention, the plasma separation unit includes a blood cell adsorbent, a blood cell adsorption material or a blood cell adsorption gel.

In yet a further separate aspect of the present invention, the plasma separation unit includes a tube to contain the plasma layer and the blood cell layer formed in the filtered blood.

Further scope of the applicability of the present invention will become apparent from the detailed description given hereinafter. However, it should be understood that the detailed description and specific examples, while indicating preferred embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a flowchart of a blood isolation and extraction method in accordance with a first preferred embodiment of the present invention.

FIG. 2 is a schematic block diagram of a blood isolation and extraction device in accordance with a first preferred embodiment of the present invention.

FIG. 3 is a flowchart of a blood isolation and extraction method in accordance with a second preferred embodiment of the present invention.

FIG. 4 is a schematic block diagram of a blood isolation and extraction device in accordance with a second preferred embodiment of the present invention.

FIG. 5 is a schematic block diagram of a blood isolation and extraction device in accordance with a third preferred embodiment of the present invention.

### DETAILED DESCRIPTION OF THE INVENTION

It is noted that a blood isolation and extraction method and device thereof in accordance with the preferred embodiment of the present invention is suitable for manufacturing various whole blood products and related products thereof, including packed blood cells, fresh frozen plasma, cryoprecipitate, platelet concentrate, leukocyte concentrate and related products thereof for example, which are not limitative of the present invention. The blood isolation and extraction method and device thereof in accordance with the preferred embodiment of the present invention can also be applicable to various medical research, medical treatment (e.g. Achilles tendon rupture or arthritis treatment), medical cosmetology (e.g. baldness, wrinkle or collagen treatment), medical rehabilitation (e.g. plastic surgery or dental implant surgery), pharmaceutical products or related industries thereof, which are not limitative of the present invention.

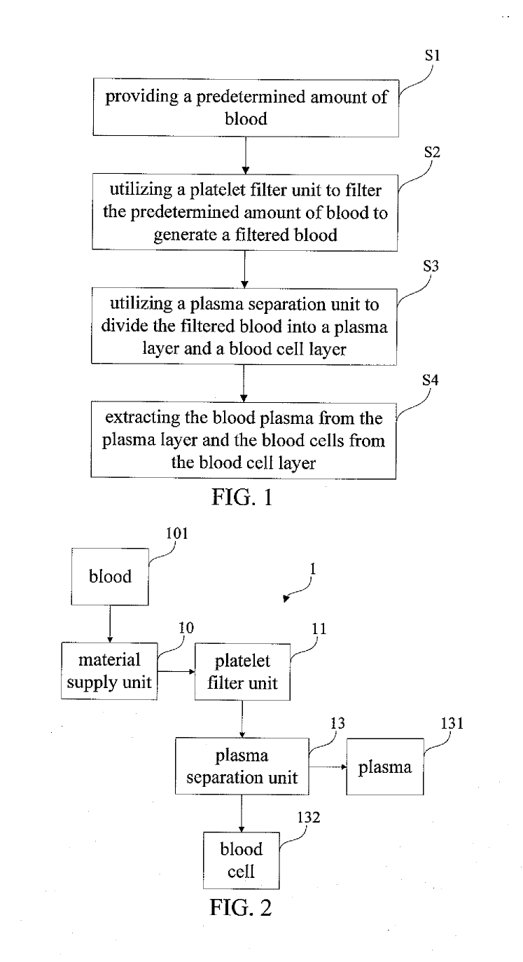

FIG. 1 shows a flowchart of a blood isolation and extraction method in accordance with a first preferred embodiment of the present invention. FIG. 2 shows a schematic block diagram of a blood isolation and extraction device in accordance with the first preferred embodiment of the present invention, corresponding to the blood isolation and extraction method shown in FIG. 1. Referring now to FIGS. 1 and 2, the blood isolation and extraction device 1 in accordance with the first preferred embodiment of the present invention includes a material supply unit 10 (e.g. pump, injector or the like), a platelet filter unit 11 (e.g. platelet filter device) and a plasma separation unit 13 (e.g. plasma separation device) which can meet gamma radiation sterilization and biocompatibility requirement.

Still referring to FIGS. 1 and 2, by way of example, the material supply unit 10 connects the platelet filter unit 11 with a first conduit or pipeline and the platelet filter unit 11 further connects the plasma separation unit 13 with a second conduit or pipeline. The material supply unit 10 is suitably installed with a blood-drawing unit 101 or other blood-collecting devices (e.g., venipuncture syringes or test tubes).

With continued to FIGS. 1 and 2, the blood isolation and extraction method in accordance with the first preferred embodiment of the present invention includes the step S1: automatically, semi-automatically or manually operating the material supply unit 10 to supply a predetermined amount of blood (i.e. whole blood) to the platelet filter unit 11. By way of example, the material supply unit 10 includes an injector, a syringe or a pump device to operate the blood-drawing unit 101 so as to supply the blood to the platelet filter unit 11. In addition, the material supply unit 10 further includes a server motor or the like to pump the blood.

With continued to reference FIGS. 1 and 2, the blood isolation and extraction method in accordance with the first preferred embodiment of the present invention includes the step S2: automatically, semi-automatically or manually operating the platelet filter unit 11 to filter the predetermined amount of blood to generate a filtered blood. By way of example, after the predetermined amount of blood is pumped to pass through the platelet filter unit 11 with a predetermined pressure, platelets are selectively adsorbed and remained on a surface portion of the platelet filter unit 11. Thereafter, the platelets will not be contained in the filtered blood. Advantageously, the blood isolation and extraction method of the present invention can avoid problems of rapid loss of filtered blood pressure, velocity drop of filtered blood flow and choke of filter holes in filtering operation.

With continued to reference FIGS. 1 and 2, by way of example, the platelet filter unit 11 includes a platelet adsorption net filter or a platelet adsorption plate. The platelet adsorbing net filter includes a plurality of apertures performed as filtering holes and is made of platelet adsorption resin or other equivalent materials. The platelet adsorption plate is also made of platelet adsorption resin other equivalent materials. The material of platelet adsorption resin has a function of platelet adsorption and a high degree of recovery of platelets.

By way of example, the platelet adsorption resin or resin is modified by a surface modifier selected from modifiers containing phosphatidyl choline (pc) derivatives or zwitterionic dopamine derivatives which have highly stable and water absorbability. The zwitterionic structure can avoid bonding with other metal bonds, polynucleotide and proteins. In addition, the zwitterionic structure has a high degree of hydrophile even though it can absorb four times of water to contain proteins after dried. Advantageously, it is hard to be permeated or absorbed to prevent clot reaction or hemolytic reaction.

With continued to reference FIGS. 1 and 2, the blood isolation and extraction method in accordance with the first preferred embodiment of the present invention includes the step S3: transferring the filtered blood to the plasma separation unit 13 and automatically, semi-automatically or manually operating the plasma separation unit 13 to divide the filtered blood into a plasma layer and a blood cell layer in a predetermined tube for separating blood cells (i.e. corpuscles) from blood plasma. Generally, the plasma layer is an upper layer (i.e. creamy yellow) relatively while the blood cell layer is a lower layer.

With continued to reference FIGS. 1 and 2, by way of example, the plasma separation unit 13 includes a blood cell adsorbent, a blood cell adsorption gel or other blood cell adsorption materials. The blood cell adsorbent or the blood cell adsorption gel is cured on sponges, foam rubber. hemostatic cotton or surfaces of other fillers which are preferably contained in a cone container.

With continued to reference FIGS. 1 and 2, the blood isolation and extraction method in accordance with the first preferred embodiment of the present invention includes the step S4: automatically, semi-automatically or manually operating a tool (e.g. pipette or other equivalent tools) to draw out the liquids of plasma layer and blood cell layer for extracting the blood plasma from the plasma layer and the blood cells from the blood cell layer. With continued to reference FIGS. 1 and 2, by way of example, the blood plasma is stored in a plasma storage unit 131 (i.e. first sterilized container or other equivalent devices) while the blood cells are separately stored in a blood cell storage unit 132 (i.e. second sterilized container or other equivalent devices).

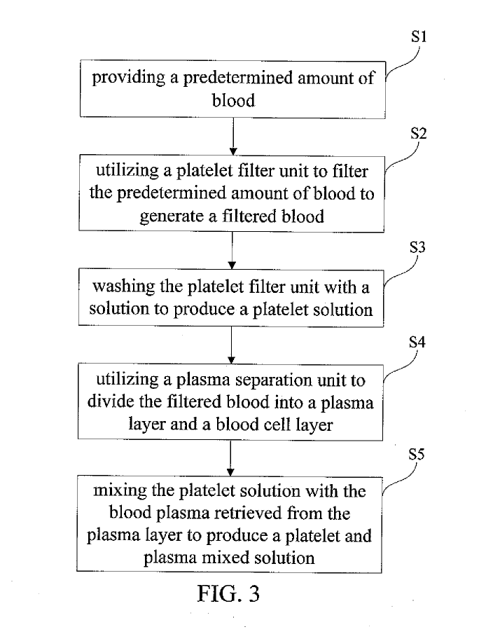

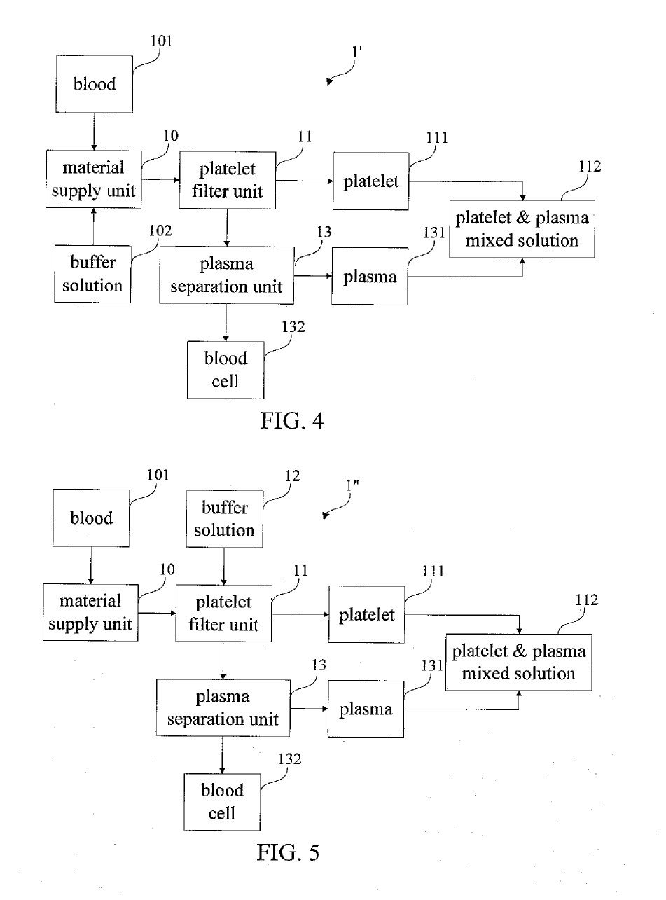

FIG. 3 shows a flowchart of a blood isolation and extraction method in accordance with a second preferred embodiment of the present invention. FIG. 4 shows a schematic block diagram of a blood isolation and extraction device in accordance with the second preferred embodiment of the present invention, corresponding to the blood isolation and extraction method shown in FIG. 3.

Referring now to FIGS. 3 and 4, the blood isolation and extraction device 1′ in accordance with the second preferred embodiment of the present invention includes a material supply unit 10, a blood-drawing unit 101, a buffer supply unit 102, a platelet filter unit 11 and a plasma separation unit 13 which can meet gamma radiation sterilization and biocompatibility requirement.

Still referring to FIGS. 3 and 4, by way of example, the blood-drawing unit 101 and the buffer supply unit 102 connect the material supply unit 10 with a first conduit, the material supply unit 10 further connects the platelet filter unit 11 with a second conduit and the platelet filter unit 11 further connects the plasma separation unit 13 with a third conduit. The material supply unit 10 is suitably installed with a blood supply source or other blood-collecting devices.

With continued to FIGS. 3 and 4, the blood isolation and extraction method in accordance with the second preferred embodiment of the present invention includes the step S1: automatically, semi-automatically or manually operating the material supply unit 10 to supply a predetermined amount of blood (i.e. whole blood) to the platelet filter unit 11. By way of example, the material supply unit 10 includes an injector, a syringe or a pump device to operate the blood-drawing unit 101 so as to supply the blood to the platelet filter unit 11. In addition, the material supply unit 10 further includes a server motor or the like to pump the blood.

With continued to reference FIGS. 3 and 4, the blood isolation and extraction method in accordance with the second preferred embodiment of the present invention includes the step S2: automatically, semi-automatically or manually operating the platelet filter unit 11 to filter the predetermined amount of blood to generate a filtered blood. By way of example, after the predetermined amount of blood is pumped to pass through the platelet filter unit 11 with a predetermined pressure, platelets are selectively adsorbed and remained on a surface portion of the platelet filter unit 11. Thereafter, the platelets will not be contained in the filtered blood. Advantageously, the blood isolation and extraction method of the present invention can avoid problems of rapid loss of filtered blood pressure, velocity drop of filtered blood flow and choke of filter holes in filtering operation.

With continued to reference FIGS. 3 and 4, the blood isolation and extraction method in accordance with the second preferred embodiment of the present invention includes the step S3: automatically, semi-automatically or manually operating the material supply unit 10 and the buffer supply unit 102 to wash or to flush the platelet filter unit 11 with a solution (or a buffer solution) at least one or several times to produce a platelet solution which contains platelets remained on the platelet filter unit 11. By way of example, the platelet solution is stored in a platelet storage unit 111 (i.e. sterilized container). In platelet washing operation, the material supply unit 10 has ceased to supply the blood from the blood-drawing unit 101.

With continued to reference FIGS. 3 and 4, by way of example, the material supply unit 10 includes an injector, a syringe or a pump device to operate the buffer supply unit 102 to form a platelet-washing unit which can wash or flush the platelets on the platelet filter unit 11 with the buffer solution (e.g. phosphate buffer solution, PBS).

With continued to reference FIGS. 3 and 4, the blood isolation and extraction method in accordance with the second preferred embodiment of the present invention includes the step S4: transferring the filtered blood to the plasma separation unit 13 and automatically, semi-automatically or manually operating the plasma separation unit 13 to divide the filtered blood into a plasma layer and a blood cell layer in a predetermined tube for separating blood cells from blood plasma. Generally, the plasma layer is an upper layer (i.e. creamy yellow) relatively while the blood cell layer is a lower layer.

With continued to reference FIGS. 3 and 4, the blood isolation and extraction method in accordance with the second preferred embodiment of the present invention includes the step S5: automatically, semi-automatically or manually mixing the platelet solution with the blood plasma retrieved from the plasma layer to produce a platelet and plasma mixed solution. By way of example, the platelet and plasma mixed solution is stored in a mixed solution storage unit 112 (i.e. sterilized container).

FIG. 5 shows a schematic block diagram of a blood isolation and extraction device in accordance with a third preferred embodiment of the present invention. Referring to FIG. 5, the blood isolation and extraction device 1″ in accordance with the second preferred embodiment of the present invention includes a material supply unit 10, a blood-drawing unit 101, a platelet filter unit 11, a platelet-washing unit 12 and a plasma separation unit 13 which can meet gamma radiation sterilization and biocompatibility requirement.

Still referring to FIG. 5, by way of example, the platelet-washing unit 12 connects the platelet filter unit 11 with a conduit. In platelet washing operation, the platelet-washing unit 12 is automatically, semi-automatically or manually operated to wash or to flush the platelet filter unit 11 with a solution (or a buffer solution) at least one or several times to produce a platelet solution. Synchronously, the material supply unit 10 has ceased to supply the blood from the blood-drawing unit 101.

Although the invention has been described in detail with reference to its presently preferred embodiment, it will be understood by one of ordinary skills in the art that various modifications can be made without departing from the spirit and the scope of the invention, as set forth in the appended claims.

What is claimed is:

1. A blood isolation and extraction method comprising:
providing a predetermined amount of blood;
utilizing a platelet filter unit to filter the predetermined amount of blood to generate a filtered blood;
utilizing a plasma separation unit to divide the filtered blood into a plasma layer and a blood cell layer for separating blood cells from blood plasma; and
extracting the blood plasma from the plasma layer and the blood cells from the blood cell layer.

2. The blood isolation and extraction method as defined in claim 1, wherein the predetermined amount of blood is supplied by a blood supply unit or an injector.

3. The blood isolation and extraction method as defined in claim 1, wherein the platelet filter unit includes a platelet adsorption net filter or a platelet adsorption plate.

4. The blood isolation and extraction method as defined in claim 3, wherein the platelet adsorbing net filter or the platelet adsorption plate is made of platelet adsorption resin.

5. The blood isolation and extraction method as defined in claim 1, wherein the plasma separation unit includes a blood cell adsorbent, a blood cell adsorption material or a blood cell adsorption gel.

6. A blood isolation and extraction method comprising:
providing a predetermined amount of blood;
utilizing a platelet filter unit to filter the predetermined amount of blood to generate a filtered blood;
washing the platelet filter unit with a solution to produce a platelet solution;
utilizing a plasma separation unit to divide the filtered blood into a plasma layer and a blood cell layer for separating blood cells from blood plasma; and
mixing the platelet solution with the blood plasma retrieved from the plasma layer to produce a platelet and plasma mixed solution.

7. The blood isolation and extraction method as defined in claim 6, wherein the predetermined amount of blood is supplied by a blood supply unit or an injector.

8. The blood isolation and extraction method as defined in claim 6, wherein the platelet filter unit includes a platelet adsorption net filter or a platelet adsorption plate.

9. The blood isolation and extraction method as defined in claim 8, wherein the platelet adsorbing net filter or the platelet adsorption plate is made of platelet adsorption resin.

10. The blood isolation and extraction method as defined in claim 6, wherein the plasma separation unit includes a blood cell adsorbent, a blood cell adsorption material or a blood cell adsorption gel.

11. The blood isolation and extraction method as defined in claim 6, wherein the solution is a buffer solution.

12. A blood isolation and extraction device comprising:
a platelet filter unit provided to filter a predetermined amount of blood to generate a filtered blood; and
a plasma separation unit provided to divide the filtered blood into a plasma layer and a blood cell layer for separating blood cells from blood plasma;
wherein the blood plasma is capable of retrieving from the plasma layer and the blood cells are capable of retrieving from the blood cell layer.

13. The blood isolation and extraction device as defined in claim 12, wherein a platelet-washing unit is further provided to wash the platelet filter unit with a solution to produce a platelet solution.

14. The blood isolation and extraction device as defined in claim 13, wherein the platelet-washing unit includes a buffer dispenser.

15. The blood isolation and extraction device as defined in claim 13, wherein the solution is a buffer solution.

16. The blood isolation and extraction device as defined in claim 12, wherein the predetermined amount of blood is supplied by a blood supply unit or an injector.

17. The blood isolation and extraction device as defined in claim 12, wherein the platelet filter unit includes a platelet adsorption net filter or a platelet adsorption plate.

18. The blood isolation and extraction device as defined in claim 17, wherein the platelet adsorbing net filter or the platelet adsorption plate is made of platelet adsorption resin.

19. The blood isolation and extraction device as defined in claim 12, wherein the plasma separation unit includes a blood cell adsorbent, a blood cell adsorption material or a blood cell adsorption gel.

20. The blood isolation and extraction device as defined in claim 12, wherein the plasma separation unit includes a tube to contain the plasma layer and the blood cell layer formed in the filtered blood.
